(19) United States
(12) Patent Application Publication
(10) Pub. No.: US 2025/0090036 A1
(43) Pub. Date: Mar. 20, 2025

(54) Multichannel Heartbeat Detection by Temporal Pattern Search

(71) Applicant: Bruce Hopenfeld, New Harmony, UT (US)

(72) Inventor: Bruce Hopenfeld, New Harmony, UT (US)

(21) Appl. No.: 18/301,218

(22) PCT Filed: Oct. 11, 2021
(86) PCT No.: PCT/US2021/054339

§ 371 Date: Apr. 15, 2023

Related U.S. Application Data
(60) Provisional application No. 63/094,539, filed on Oct. 21, 2020.
(60) Provisional application No. 63/140,939, filed on Jan. 24, 2021.

(51) Int. Cl. A61B 5/0245; A61B 5/352

(52) U.S. Cl. CPC A61B 5/0245; A61B 5/352

(57) ABSTRACT

A method for detecting heart beats within multichannel cardiac signals is disclosed. A plurality of sensors are configured to receive multiple channel cardiac signals. A processor is configured to preprocess the cardiac signal and then detect heart beats by separately analyzing signal segments. Separately in each channel, candidate peaks are selected. Peaks across channels that are closely time aligned are merged, and a peak timing coherence probability is computed for each merged peak. A sequence search is then performed on global peaks, which comprise the merged peaks and the remainder of the candidate peaks (which exist only in a single channel). Resulting sequences are assigned raw scores based on: 1) the sum of the sequence's peak coherence probabilities; 2) the sum of peak pair prominence scores across all channels; 3) rhythm probability, which is temporal regularity in the case of sinus rhythm; and 4) the number of skipped beats.

### PRIORITY CLAIM

This application claims priority to: U.S. Provisional Patent Application Ser. No. 63/094,539, filed on Oct. 21, 2021, and U.S. Provisional Patent Application Ser. No. 63/140,939, filed on Jan. 24, 2021.

### TECHNICAL FIELD

The invention pertains primarily to cardiac monitoring, and in particular heartbeat detection in single or multi-channel cardiac signals such as electrocardiograms.

### BACKGROUND ART

The continued evolution of wearable sensors and portable electronics will likely result in the substantial growth of long term day-to-day cardiac monitoring. Important aspects of cardiac monitoring include the estimation of heart rate and the detection of atrial fibrillation. Despite the advances in wearable sensors, noise remains a significant problem. Relatedly, there are a variety of methods for combining multiple channels, which may or may not correspond to different recording modalities, but none of these has been proven optimal.

U.S. Pat. No. 9,402,557, issued Aug. 2, 2016 to the inventor of the present invention, describes systems and methods for detecting sequences of heartbeats in noisy signals by performing combinatorial optimization on peak temporal regularity within a single channel. This single channel work was extended so that sequences were also scored according to a relative signal-to-noise, peak prominence measure; this extension is prior art.

Tejedor et al. describe four main approaches for fusing multiple channels which often correspond to signals from different recording modalities: 1) RR-based methods; 2) signal switching; 3) voting; and 4) approaches that merge heartbeat detection and fusion into a single step. Tejedor, J.; Garcia, C. A.; Márquez, D. G.; Raya, R.; Otero, A. “Multiple Physiological Signals Fusion Techniques for Improving Heartbeat Detection: A Review.” Sensors 2019, 19, 4708.

An example of an RR-based method is disclosed by Antink, who describes a Bayesian approach to estimate the probability that RR intervals associated with two different channels correspond to two consecutive true heartbeats. Antink, C. H., Gao, H., Brüser, C., & Leonhardt, S. (2015). “Beat-to-beat heart rate estimation fusing multimodal video and sensor data.” Biomedical optics express, 6(8), 2895-2907. The Bayesian estimator (for a particular RR interval) is obtained by multiplying together the autocorrelation Bayesian probabilities of separate channels. This type of autocorrelation will tend to struggle in high noise conditions where noise peaks and true beats have similar sizes/shapes.

Signal switching selects heartbeats from a high-quality channel from amongst a number of channels. This technique fails to utilize information in channels that are not selected. Unlike signal switching techniques, voting techniques combine information from multiple channels but they do so in a suboptimal way. An example of a voting technique is described by Yu et al (Yu, Q., Guan, Q., Li, P. et al. “Fusion of detected multi-channel maternal electrocardiogram (ECG) R-wave peak locations.” BioMed Eng OnLine 15, 4 (2016)). Conventional peak detection is separately performed in each of a number of channels; peaks that are detected in less than (or equal to) one half of the channels are removed. However, conventional peak detection will tend to fail in high noise conditions where noise peaks and true beats have similar sizes/shapes. Bernd Porr, Luis Howell, “R-peak detector stress test with a new noisy ECG database reveals significant performance differences amongst popular detectors.” bioRxiv preprint 2019, 722397.

An example of Tejedor's category number 4, approaches that merge heartbeat detection and fusion into a single step, is described by Zia, T.; Arif, Z. “Probabilistic data fusion model for heartbeat detection from multimodal physiological data.” Turk. J. Electr. Eng. Comput. Sci. 2017, 25, 449-460. A Bayesian scheme is applied to correlate an ECG signal with a blood pressure signal in such a manner to detect heartbeats. A heartbeat is signified when a Bayesian fusion classifier exceeds a threshold.

Much of the above-mentioned work involves attempts to solve the problem of correlating signals that have very different heartbeat shapes. For example, an ECG heartbeat waveform is much more localized than a blood pressure heartbeat waveform. In the case of ECG signals corrupted with certain types of noise, such as motion artifact noise, heartbeat and noise waveforms are frequently indistinguishable. When one channel is such a noisy ECG signal, any fusion scheme that relies on a simple threshold to detect heartbeats will result in many false positives (if ECG detection criteria are broad) or false negatives (if ECG detection criteria are narrow).

At least three groups have applied neural networks to multiple channel electrocardiograms: Antink, C. H.; Breuer, E.; Uguz, D. U.; Leonhardt, S. “Signal-Level Fusion with Convolutional Neural Networks for Capacitively Coupled ECG in the Car.” 2018 Computing in Cardiology Conference (CinC) 2018, 45, 1-4; Ravichandran, V.; Murugesan, B.; Shankaranarayana, S.; Ram, K.; Preejith, S. P.; Joseph, J.; Sivaprakasam, M. “Deep Network for Capacitive ECG Denoising.” arXiv preprint 2019, 1903.12536v1; Chandra, B. S.; Sastry, C. S.; Jana, S. Robust heartbeat detection from multimodal data via CNN-based generalizable information fusion. IEEE Trans. Biomed. Eng. 2018, 66, 710-717. Neural networks often do not generalize well; it is not clear how well these architectures would work across a variety of high noise multi-channel datasets. Also, to reduce the neural network parameter space, temporal resolution tends to be poor.

### DISCLOSURE OF INVENTION

The present invention overcomes the limitations of the multiple channel prior art by searching for temporal patterns in multiple cardiac signals by performing combinatorial optimization on a set of peaks derived from all channels. Multiple sequences are generated from the global set, these sequences are scored according to quality measures, and a winning sequence is chosen. The quality measures preferably include a peak timing coherence measure that discriminates between peak time differences of true beats and noise peaks. The peak timing coherence measure takes advantage of the information associated with sharply localized peaks in certain types of signals such as electrocardiograms.

Specifically, a plurality of sensors are configured to receive multiple channel cardiac signals. A processor is configured to preprocess the cardiac signal and then detect heartbeats by separately analyzing signal segments. Separately in each channel, peak detection is performed, and candidate peaks are located based on peak prominence. Peak pair prominence scores are computed for the candidate peaks.

Peaks across channels that are closely time aligned are merged, and a peak timing coherence probability is computed for each merged peak. A search is then performed on the global peaks, which comprise the merged peaks and the remainder of the candidate peaks (which exist only in a single channel). The search is performed by selecting high quality global peaks, generating parent sequences from the global peaks, and then generating offspring sequences by filling in gaps in the parent sequence with the remainder of the (lower quality) global peaks. The high quality peaks are selected according to a peak score that is based on the peak timing coherence probability and the peak prominence probability.

Offspring sequences are assigned raw scores based on: 1) the sum of the sequence's peak timing coherence quality measures; 2) the sum of peak pair prominence measures across all channels; 3) rhythm probability, which is temporal regularity in the case of sinus rhythm; and 4) the number of skipped beats.

The final scores of the offspring sequences are a weighted sum of the raw score and the raw scores of temporally matching sequences in adjacent segments.

### BRIEF DESCRIPTION OF DRAWINGS

FIG. 1 shows one possible context within which the present invention may be carried out.

FIG. 2 is a high level flow chart of a heartbeat detection and rhythm detection method based upon searching for and scoring sequences of peaks in a segmented multi-channel signal.

FIG. 3a is a flow chart of the method for finding and scoring sequences of peaks in a segmented multi-channel signal. FIG. 3b shows waveforms that illustrate some of the steps described with reference to FIG. 3a.

FIG. 3c is a flowchart of the preferred sequence generation/search procedure in the case of sinus rhythm.

FIG. 4 is a flow chart of the routine that assigns a final score to a sequence.

FIG. 5 is a flow chart of the global peak selection process described with reference to blocks 304-310 in FIG. 3.

FIG. 6 is a flow chart of the routine that assigns timing coherence probabilities to peaks.

FIG. 7 is a flow chart of the process for computing a sequence's raw score.

FIG. 8 is an image of peaks, mapped by size/quality into circles, that may be used to train an image based neural network to perform peak detection according to the teachings of the present invention.

FIG. 9 is a block diagram of a convolutional neural network based implementation of the present invention.

FIG. 10 is a block diagram of a feature extraction based partial neural network implementation of the present invention.

### BEST MODE FOR CARRYING OUT THE INVENTION

Matlab and set notation are generally used herein.

As used herein, a “peak” is a fiducial point within a portion of a cardiac signal. A raw cardiac signal may be processed through any number of differencing filters which correspond to potentially different maxima, minima or zero-crossings. Any of these different maxima, minima or zero-crossings, or functions (e.g. linear combinations) thereof, can be a “peak.”

“Physiologically” permissible or impermissible refers to possible RR intervals associated with whatever animal is being monitored. “Physiologically incompatible” sequences means that, for two physiologically permissible sequences, there is at least one peak in one of the sequences that, if added to the other sequence, would render the other sequence physiologically impermissible.

A “local peak” refers to a peak with an associated time in a single channel. A “global peak” refers to a peak that has an associated time that will be considered the detected time for a heartbeat. In the preferred embodiment, the “global peak” time corresponding to local peaks that occur close in time across channels is the mean of the local peak times. These are “merged peaks.” In the preferred embodiment, the “global peak” time corresponding to a local peak that does not occur close in time to any peak in another channel is the local peak time.

Temporally successive local or global “candidate peaks” may be separated by physiologically impermissibly short time periods.

“RR interval” refers to the time between sequence elements.

The present invention will generally be described with reference to electrocardiographic signals. However, the principles herein are directly applicable to any type of cardiac signal that is characterized by having at least one distinct peak for each cardiac cycle.

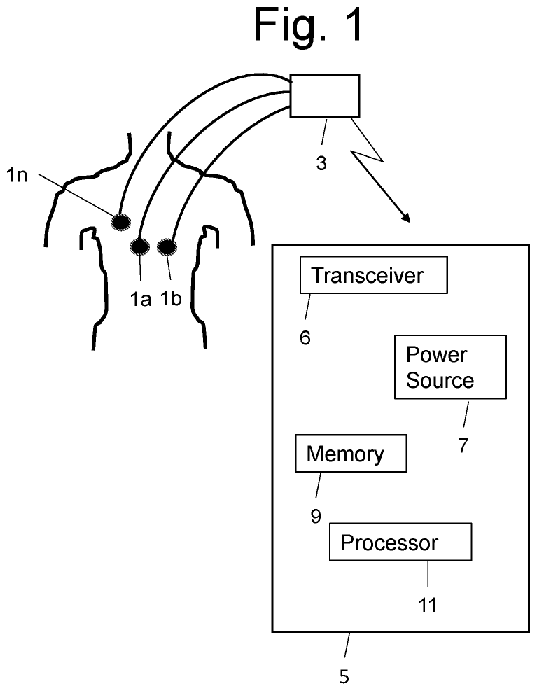

FIG. 1 shows one possible context within which the present invention may be carried out. A plurality of sensors, electrodes 1a, 1b . . . 1n, are disposed on the torso of a human body. The present invention is not limited to humans. The electrodes 1a-1n are connected to a recorder 3, which records the voltage signals therebetween. The recorder 3 may implement the cardiac analysis features of the present invention, or it may wirelessly transmit the recorded signals, after varying degrees of amplification, filtering and other processing, to a monitor 5. The monitor 5 comprises a transceiver 6 that receives signals from the recorder 3. The signals are stored in a memory 9, and processed according to the methods described herein by the processor 11. A power source 7 provides energy to the monitor 5. More details on various hardware components associated with recorder 3 and processor 5 may be found in Chen et al., “Body Area Networks: A Survey,” Mobile Networks and Applications, April 2011, Volume 16, Issue 2, pp 171-193, which is incorporated by reference herein. In no way is the present invention limited to body area networks.

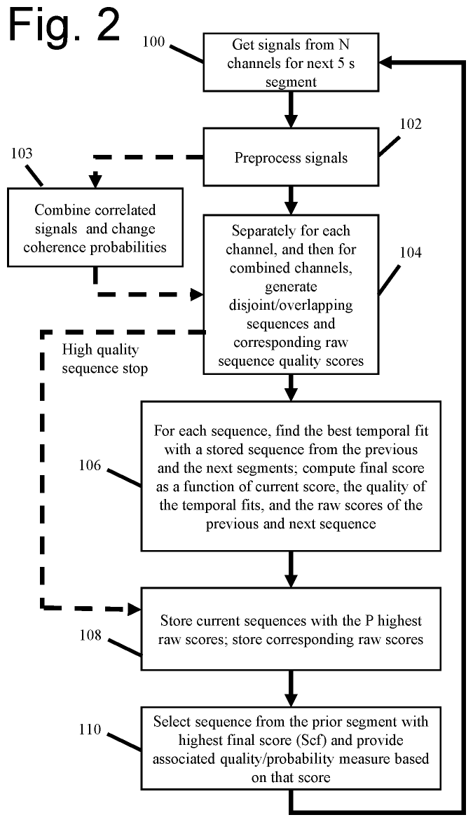

FIG. 2 is a high level flow chart of a heartbeat detection and rhythm detection method based upon searching for and scoring sequences of peaks in a segmented multi-channel signal. Each segment is preferably 5 seconds long, although segment length may increase or decrease for low or high heart rates respectively. In block 100, digital signals are acquired from N channels corresponding to electrodes 1a . . . . In in FIG. 1. In block 102, the signals are preprocessed by low pass filtering with a cutoff of 45 Hz and resampling to a rate of 256 samples/second.

Some types of sensors and configurations thereof may result in correlated signals, which can confound the temporal peak probability measures hereinafter described. One method for handling correlated signals is to add them together, as shown in block 103, averaging them according to their amplitudes. Also, as will be further described below, peak timing coherence probabilities are adjusted to reflect the correlation.

In block 104, separately for each channel, possible heartbeat peaks are detected and disjoint/overlapping sequences formed therefrom unless and until a high quality sequence is found, in which case control passes to block 108, as indicated by the dashed line. Single channel sequence quality is assessed according to the peak pair prominence score, which will be further described below, and temporal regularity criteria (different than that described below) that require that all changes in RR intervals are less than a generous threshold, e.g. 100 ms. Absent finding a high quality sequence, possible peaks across all channels are grouped together and candidate heartbeat sequences formed therefrom. A raw score is assigned to all sequences, the single channel and combined channel sequences.

In block 106, for the stored sequences from the prior segment, the best temporal fit is found with a stored sequence from its prior segment and the current segment. For 5 second segments, the temporal fit is the temporal regularity score (defined below) of a merged 5 second segment formed from the second and first 2.5 second subsegments respectively of consecutive segments. The final score of a sequence is equal to its raw score plus the raw scores of the previous and next sequences weighted by the qualities of the temporal fits.

In block 108, the current segment's P sequences with the highest raw scores are stored. The raw scores are also stored. In block 110, the sequence from the prior segment with the highest final score is selected. If its score exceeds a detection threshold, it is taken as a sequence of true heartbeat times.

For sinus rhythm, if no sequences' final scores from a segment exceed the detection threshold, a number of scores may be stored, and an optimal path (selected sequences) through the segments may be determined by maximizing (e.g. by the Viterbi algorithm) the path probability based on sequence scores and segment to segment changes in RR interval. Smaller changes in RR intervals are associated with higher probabilities.

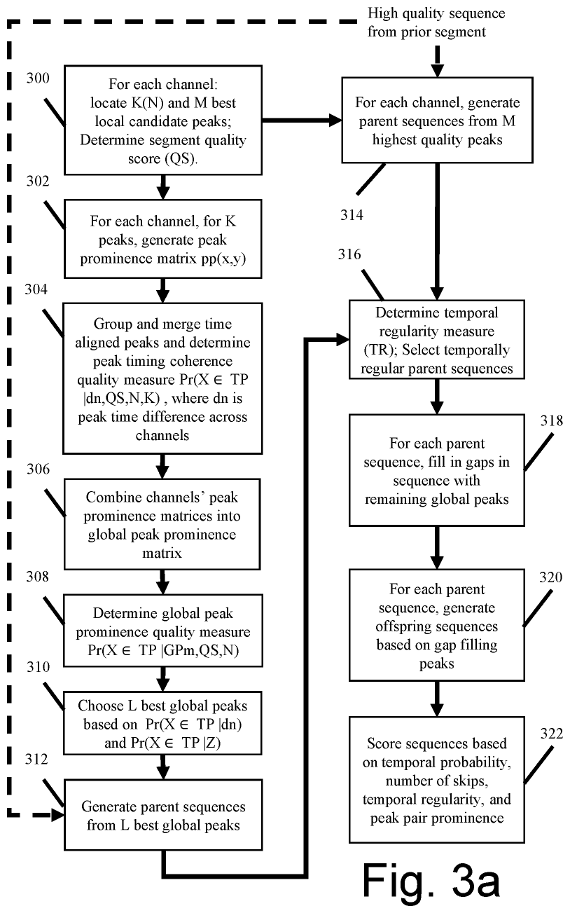

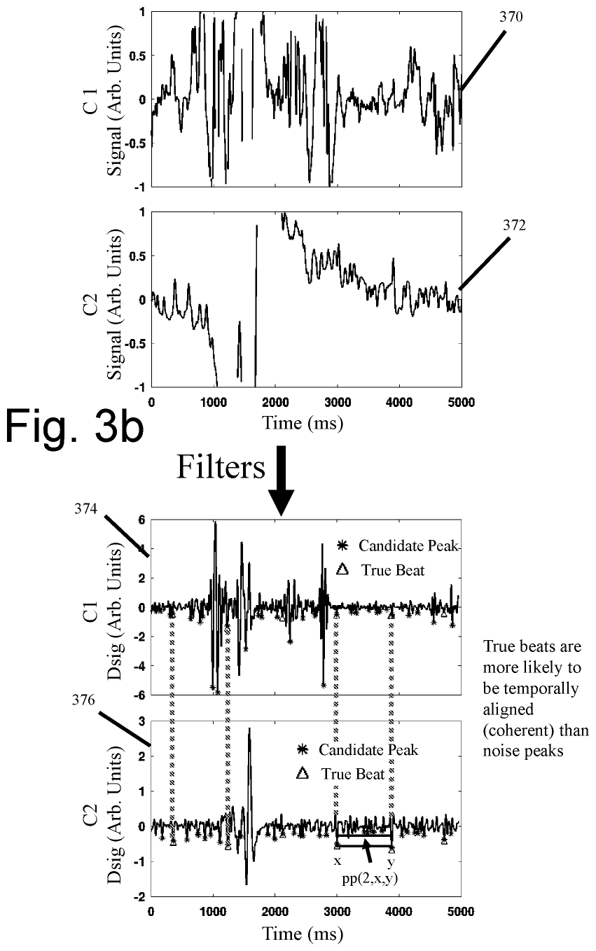

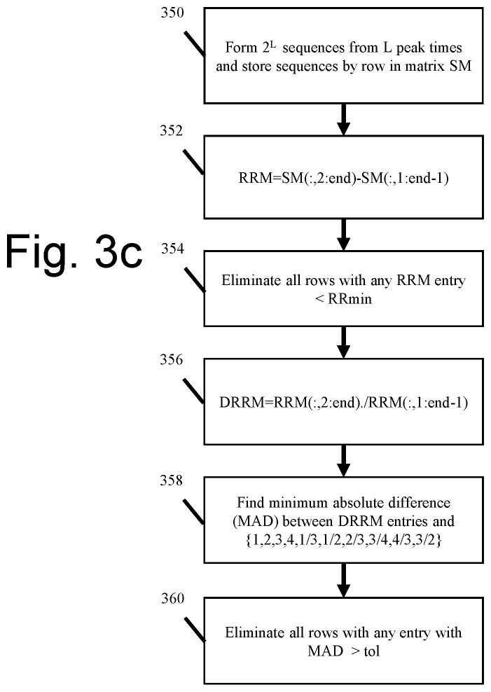

FIG. 3a is a flow chart of the method for finding and scoring sequences. In block 300, for each of the N channels, the K and M best candidate peaks are located. In many circumstances, such as those shown in FIG. 3b, which shows actual waveforms which have been processed by the present invention, successive members of these candidate peak sets may be separated by a physiologically impermissible period, which means that overlapping/disjoint sequences may be generated therefrom according to the teachings of the present invention. (Overlapping/disjoint sequences can also result from candidate peak sets that do not contain successive members separated by a physiologically impermissible period.) The value of K preferably increases with the number of channels as indicated by the reference to K(N) in block 300. (For ease of description K(N) will hereafter simply be referred to as K.) For 5 second long segments, exemplary values for K vary linearly from 20 to 40 for 1 to 4 channels.

For a signal SIG resampled to 256 samples/second, a second difference signal SIGD is generated from SIG as follows: SIGD=SIG(n−12)−2*SIG(n−6)+SIG(n). The coefficients 12 and 6 may be adjusted to maximize a segment quality score QS, which will be further described below. Plots 374 and 376 in FIG. 3b show filtered and differenced versions of raw signals shown in plots 370 and 372. Positive and negative SIGD peaks that meet minimum and maximum peak width requirements are selected. For purposes of discussion, it will be assumed that heartbeat peaks are negative dominant in SIGD. The negative polarity is arbitrarily chosen. Also, the signal polarity may be adaptively reversed to maximize the segment quality score QS. The scaling of the second difference is preferably adaptively chosen to maximize QS.

Peak prominence measures for initial peak selection are determined as follows. (This peak prominence measure is different than the peak prominence measure at the sequence level, which will be further described below.) Let the set PA={PA1, PA2 . . . } denote the amplitudes of the time ordered set of acceptable negative peaks in SIGD. The prominence of peak i is the second difference PA(i−1)−2*PA(i)+PA(i+1). (For the first and last peaks in the set, the first difference with the closest set member is doubled.) The set PAP comprises these prominence values ordered with the largest values first. The candidate peaks are selected by taking the peaks with the K largest (most negative) prominences.

A subset of the candidate peaks is selected by analyzing the distribution of the set PAP. If PA varies smoothly, the segment is likely to be of low quality. Conversely, if there are a group of peaks with similar, large PA values that are widely separated from the remaining PA values, it is likely that the segment is of high quality, and the peaks with large PA values, which form a subset of the candidate peaks, are taken as high quality peaks. As will be described below, the high quality peaks form a search set for a single channel, whereas the superset of candidate peaks is combined with the candidate peaks from other channels.

The high quality peaks are defined by a simple rule based on the largest drop in PA values over a certain number of peaks, e.g. PAP(i)−PAP(i−6) is the drop in prominence over six peaks in PAP. All M peaks with PAP values above a specified fraction of this drop are the high quality peaks. (The value M is therefore not fixed but instead depends on the characteristics of the segment being analyzed.)

The segment quality is related to the size of the PAP drop relative to slopes of the PAP curves above and below the drop. One method for obtaining a numerical value for segment quality (QS in block 300) is to train a neural network on PAP sets with known quality values, which are based on the number of heartbeat peaks in the set compared to the number of noise peaks in the set. (Instead of the above mentioned procedure, the neural network may also be used to select high quality peaks.)

In block 302, the peak prominence/SNR matrix (pp(i,x,y)) is computed separately for each channel i. For each pair of physiologically possible peaks x and y within a segment (i.e. the time between the peaks is between minimum and maximum allowable RR interval values), the corresponding matrix entry pp(i,x,y) (with peak x indexed by row and peak y indexed by column), is ratio of the mean of the peaks' amplitudes (PA(x)+PA(y))/2 to the mean of the maximum of the 5 largest peaks (in PA terms) therebetween. (If peak polarity is not considered, then PA is set equal to the difference between the maximum positive and negative values of the peak.) An example of this ratio is shown in FIG. 3b for peaks x and y in channel 2: the ratio is the amplitude represented by the lower bar connecting x and y and the upper bar representing the average amplitude of the five peaks between x and y.

In block 304, peaks that are closely time aligned across channels are grouped and merged. Each peak time is preferably set as the time of occurrence of the maximum value (at a given polarity) of a peak. Time aligned peaks are separated by dashed lines in plots 374 and 376 in FIG. 3b. A merged peak is a peak that occurs within a certain period (e.g. 24 ms=6 samples) in at least two channels. The peak time of a merged peak is the mean of the local peak times in the individual channels. The local peak time is defined as the time/sample number of the maximum negative amplitude of the differenced signal. Merged peaks are global peaks.

In an alternate embodiment, the peak time of a merged peak is a weighted average of the local peak times, where the weights are an increasing function of peak quality. For this purpose, peak quality is assessed by peak prominence and, if there are more than 2 peaks (N>2) to merge, by a clustering analysis, such that peaks that are relatively closer together are higher quality.

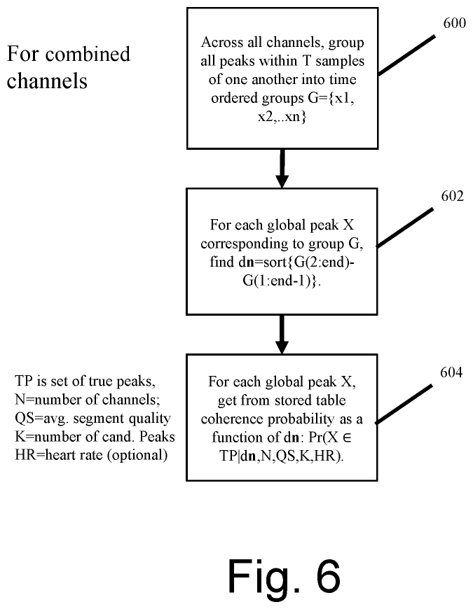

All other candidate peaks that are not merged peaks are also global peaks whose peak times are simply the peak time in the pertinent channel. Block 304 also computes a peak coherence quality measure, which is the Bayesian probability of peaks Pr(X∈TP|dn,QS,N,K), where TP indicates a true heartbeat peak, and dn is an ordered set of the time differences between peak times across channels. In an alternative embodiment, the peak time coherence is based not merely on peak time, but on a correlation between signal shapes; different peak shapes can be cross correlated. The peak shapes may correspond to different recording modalities which may be much less localized than ECG peaks such as in the case of quasi-sinusodial PPG or blood pressure peaks. The cross-correlated waveform may itself be assigned a Bayesian quality measure by comparing it to cross-correlated waveforms generated by adding controlled amounts of noise to known signals analogous to the procedure described below with reference to block 604 of FIG. 6.

In block 306, a global peak prominence matrix is then generated by summing the individual channels' matrices pp(i,x,y). In block 308, a measure of the peak prominence quality of global peaks is determined by taking the maximum values across the rows and columns of the global peak prominence matrix PP(X,Y).

In block 310, a global peak quality measure is set equal to the union of Pr(X∈TP|Z,QS,N) and Pr(X∈TP|dn,QS,N,K). (The latter probability is 0 for non-merged global peaks.) The L best peaks are selected and, in block 312, parent (sub)sequences generated therefrom. The preferred sequence generation scheme will be described with reference to FIG. 3b.

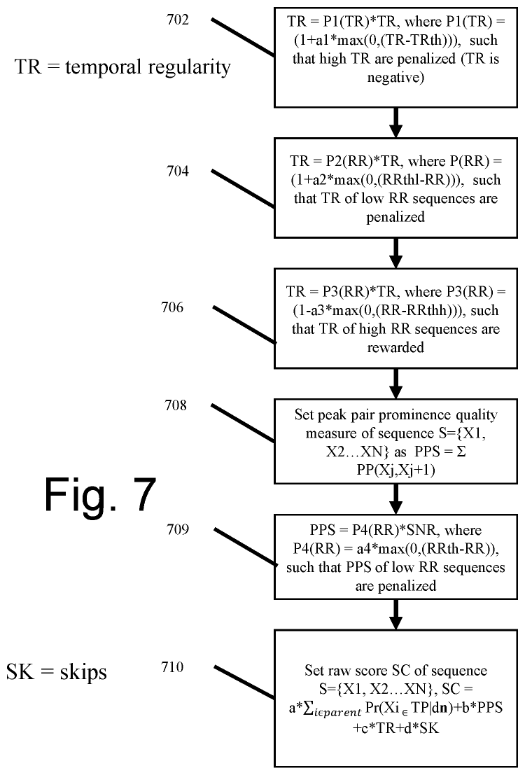

Block 300 also branches to block 314, which generates additional parent sequences separately for each channel. The M highest quality peaks are the search set. As above, the shortcut search is performed if a high quality prior sequence exists. In block 316, a temporal regularity score TR is computed for each parent sequence. The temporal regularity score is the sum of the probabilities associated with each change in RR interval in a sequence, divided by the number of changes in RR interval in a sequence. The probability for RR interval changes may be obtained from population wide data or may be specific to a particular person if such data is available. This probability is a function of the mean RR interval (lower RR intervals tend toward greater regularity) although in the preferred embodiment, as will be described with reference to FIG. 7, instead of using different probability distributions for different RR intervals, linear functions were applied to decrease the regularity score of low RR interval segments (e.g below 550 ms) and to increase the regularity score of high RR interval segments (e.g. above 850 ms).

Skipped beats are handled as follows. First, probability distributions are generated for skipped beats by randomly eliminating specified numbers of beats from the data being used to generate the temporal regularity probability distribution. For example, in the sequence [1 600 1210 1830], 600 may be randomly removed, leaving RR intervals of [1209 620]. The change in RR interval is taken as 2*620−1209=31. For example, in the sequence [1 600 1210 1830], 600 may be randomly removed, leaving RR intervals of [1209 620]. If the RR intervals are instead [1210 1850], the change in RR intervals is taken as 1850−(3/2)*1210=35. More generally, the RR interval associated with the smaller number of skips is scaled up to the larger number of skips by the fraction (skips1+1)/(skips2+1), where skips1>skips2. The standard deviation of a distribution increases with the increasing number of skips.

Parent sequences whose regularity scores exceed a threshold are passed on to block 318, which attempts to fill in the gaps in the parent sequences with the remaining global peaks. In block 320, these gap filling peaks are used to generate offspring subsequences. If there are multiple gaps in a parent sequence, and multiple peaks that fit within the gaps, combinations of possible subsequences are generated from these gap filling peaks. For example, if a first gap may be filled by peaks [500, 510] and a second gap may be filled by peaks [1501, 1511], four subsequences are generated: [500, 1501], [500, 1511], [510,1501], and [510,1511]. Each of the final offspring sequences comprises the parent sequence combined with one of the gap filling subsequences.

In block 322, sequences are scored based on peak timing coherence quality, number of skips, temporal regularity, and peak pair prominence quality, as will be further described with reference to block 710 (FIG. 7).

FIG. 3c is a flowchart of the preferred sequence generation/search procedure invoked by blocks 312 and 314 in FIG. 3a. In block 350, all (2^{L}) possible sequences are formed from L peaks and stored in matrix SM. In block 352, the RR interval matrix RRM is formed, and in block 354, sequences with consecutive members that are separated by less than the minimum possible RR interval are eliminated. For sinus rhythm, in block 356, the ratios of all consecutive RR intervals are computed for each sequence. In block 358, the absolute value of the difference between these ratios and a list of allowable integers/fractions is computed. If up to 3 skipped beats are allowed, the allowable integers/fractions are: {1 2 3 4 ¼ ⅓ ½ ⅔ ¾ 4/3 3/2}. In block 360, the ratio is accepted if the absolute value is less than a threshold, e.g. 0.3. A sequence is accepted only if all of its ratios are accepted. The acceptable sequences are deemed parent sequences; offspring sequences will be generated by attempting to find beats that fill in skipped beats.

If a high-quality sequence from the prior segment is available, the search procedure is constrained accordingly. The last peak in the prior segment sequence (assuming no skips) serves as the origin (offset time) for the peak times in the current segment search set. After this offset is added to the current peaks, they are divided by the last RR interval in the prior sequence. Peaks whose corresponding remainder is close to an integer are candidate peaks that possibly continue the prior sequence. If a sufficient number of such peaks exist, parent sequences are generated therefrom to the exclusion of the parent sequence generation described above. The more exhaustive search may still be carried out if the prior sequence based shortcut search does not result in a high quality sequence.

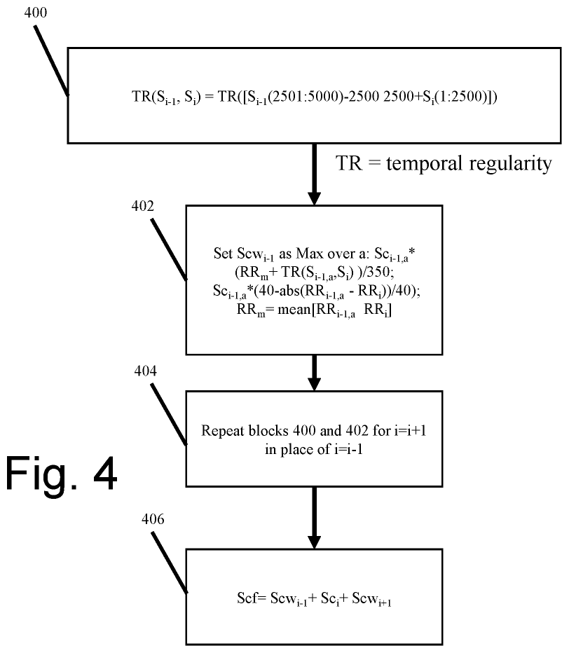

FIG. 4 is a flow chart of the routine that assigns a final score to a sequence. In block 400, the temporal match between a sequence at time i and a sequence at time i−1 is found by forming an overlapping sequence comprising the last half of the i−1 sequence with the first half of the sequence at time i: [Sᵢ₋₁(2501:5000)−2500 2500+Sᵢ(1:2500)]), where Sᵢ₋₁(2501:5000) are the peaks after 2500 ms and before 5000 ms in the sequence Sᵢ₋₁ and Sᵢ(1:2500) are the peaks after Oms and before 2500 ms in the sequence Sᵢ. The regularity score Regsc(Sᵢ₋₁, Sᵢ) (block 316) is then assigned to this overlapping sequence.

In block 402, the overlapping regularity score is weighted with the raw score of the prior sequences Sc(Sᵢ₋₁,a), where a is an index into the stored prior (t=i−1) sequences. Also, in case there isn't a good peak by peak temporal match, the RR interval match abs(RRᵢ₋₁,a−RRᵢ) is computed, and the raw score Sc(Sᵢ₋₁,a), is also weighted by the RR interval match. Over all of the prior sequences indexed by ‘a’, the maximum raw score, weighted separately by peak by peak and RR interval matches, is chosen. (RRᵢ₋₁,a denotes one of the prior sequences indexed by ‘a’ while RRᵢ indicates the current sequence).

In block 404, blocks 400 and 402 are repeated for the sequences from the next segment (i+1) instead of the prior segment (i−1). The overlapping sequences become [Sᵢ(2501:5000)−2500 2500+Sᵢ+(1:2500)] but otherwise i−1 replaces i+1.

In block 406, the final score for the current sequence is its raw score plus the maximum weighted scores associated with the prior and subsequent segments as computed in blocks 400 and 402. (The “current” segment i with reference to FIG. 4 is the “prior” segment in FIG. 2.)

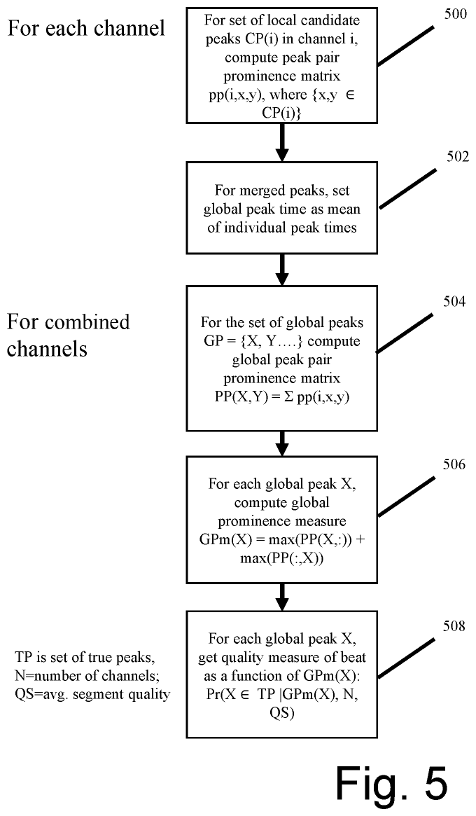

FIG. 5 is a flow chart of the global peak selection process described with reference to blocks 304-310 in FIG. 3a. For the set CP(i) in channel i of the K candidate peaks in that channel, the peak pair prominence matrix pp(i,x,y) is generated, where {x,y∈CP(i)}. As previously described, the peak prominence entry pp(x,y) is the mean of the peak magnitudes of x and y, divided by the mean of the 5 largest peaks in between x and y. (Peak magnitude refers to the maximum of a peak of the differenced signal described with reference to block 102, FIG. 2. “Largest” refers to peak magnitude.)

In block 502, the global peak time for merged peaks, which are peaks that occur within 6 samples (sampling rate 256 Hz) of one another across channels, is set equal to the mean of the peak times in the individual channels. The rest of the set of global peaks is the union the candidate peaks across all channels that are not merged peaks. In block 504, the global peak pair prominence matrix entry PP(X,Y) is set equal to the sum over channels E pp(i,x,y) of the individual peak prominence entries corresponding to the global peaks X and Y. (If either X or Y is a single channel peak, the global PP entry is equal to the pertinent single channel entry.) For example, if a peak occurs at 1000 and 1002 ms in channels 1 and 2 respectively, it is classified as merged peak. Likewise with a peak that occurs at 1600 and 1596 in the two channels. If the pair prominence between peak 1000 and 1600 in channel 1 is A=pp(1,1000,1600), and the pair prominence between peak 1002 and 1599 in channel 2 is B=pp(2,1002,1596), the pair prominence for global/merged peak 1001 (=mean(1000,1002)) and global/merged peak 1598 (=mean(1600,1596)) is A+B. The global peak prominence/SNR matrix is designated by PP(X,Y), where X, Y are global peaks (1001 and 1598 in the above example).

In block 506, for each global peak X (which isn't necessarily a merged peak), a global prominence measure GPm(X)=max(PP(X,:))+max(PP(:,X)) is computed. GPm(X) is used in block 508 to obtain, for each global peak X, a peak prominence quality measure, which in this case is the Bayesian probability of a true beat as a function of peak prominence: Pr(X∈TP|GPm(X), N, QS). The probability Pr(X∈TP|GPm(X),QS,N) may be estimated by running simulations by adding calibrated amounts of noise to a known signal for various numbers of channels, and the percentage of true peaks tabulated for given values of GPm(X), QS, and N. The electromyogram noise record from the Physionet Noise Stress Test was used as the noise source added to a simulated clean lead I or II like waveform whose RR intervals were taken from the Physionet Normal Sinus Rhythm Database. The QS was computed for each simulated segment since this is the quantity (not the a priori “noise level”) that can be computed for a real world data segment (block 300 of FIG. 3).

FIG. 6 is a flow chart of the routine that determines timing coherence probabilities for peaks. In block 600, across all channels, all peaks within T samples of one another are grouped into time ordered sets G={x1, x2, . . . xn}. T is 6 samples at a sampling rate of 256 Hz. In block 602, the (ascending) sorted difference in samples between successive members of G is stored in vector dn.

In block 604, dn is used to obtain the a posteriori coherence probability of peak X Pr(X∈TP|dn). For two channels, dn is a single number, and this Bayesian probability is equal to (Pr(dn|X∈TP)*Pr(T))/(Pr(dn|X∈TP)*Pr(X∈TP)+Pr(dn|NS)*Pr(X∈NS)), where P(dn|X∈TP) is the probability of heartbeat peaks being measured within dn samples across two channels, Pr(dn|X∈NS) is the probability of noise peaks being measured within dn across two channels and P(X∈TP) and Pr(X∈NS) are the prior probabilities of true and noise peaks respectively. In the case of randomly distributed noise, within a 5 second signal segment, the probability Pr(dn|X∈NS) of two noise peaks in respective channels occurring within a certain time dn is a function of the number of noise peaks. (More abstractly, noise peak temporal density governs the probability. Also, if one peak is a true beat but the other is a noise peak that has obliterated a true peak, the noise peak is considered a true peak.) Since the total number of selected peaks in a segment includes an unknown number of heartbeat peaks and noise peaks, neither the priors Pr(X∈TP), Pr(X∈NS) nor the likelihood Pr(dn|X∈NS) is known. (If a close in time prior segment had a high QS, an estimate of the number of true beats is available but the number that occur in the set of K candidate peaks is a function of the noise level.)

The probability Pr(X∈TP|dn,QS,N,K) can be estimated by running simulations by adding varying amounts of noise to a multiple channel clean signal whose peak times are known, and the percentage of true peaks tabulated for given values of dn, QS, N, and K. The noise should vary over a range from nothing to the point where the true signal is effectively obliterated in the corrupted signal. (In an alternative embodiment, the probability could be further conditioned on heart rate, which could be estimated in real time if a high-quality estimate of RR interval is available from a prior segment. In this case, the simulations would be carried out over various heart rates.) QS is the segment quality (averaged across channels) as previously described, K is the number of candidate peaks as previously described, and dn is the time ordered set of the minimum peak time differences across channels. For example, if peaks occur at samples 100, 103, and 105 in channels 1 through 3 respectively, dn={2,3}. The estimates Pr(X∈TP|dn,QS,N,K) are stored in a lookup table/matrix.

From the simulation data, it is possible to separate the prior probabilities from the likelihoods to obtain separate estimates of the distributions. This is not done in the preferred embodiment but could be useful if there is an external source of information that separately influences these distributions.

It is also possible to estimate Pr(X∈TP|dn,QS,N,K) based on theoretical assumptions and/or by simulations with simulated noise peak times.

The dn values may need to be adjusted for inherent peak time differences across channels. These inherent differences may be determined when segments in the pertinent channels are relatively clean (high QS).

Alternatively, the differences may be determined by attempting to separately find high scoring sequences in the pertinent channels and minimizing the time difference between these sequences. (The scoring of a sequence in an individual channel excludes peak time coherence probability measures.) In another alternative embodiment, then differences could be determined by a cross correlation analysis of the peak times of the candidate peaks across two channels.

In a system calibration phase, peak times may also be measured with the various sensors 1a-1n in different, known locations. During normal system operation, sensor location may be determined in a variety of ways, and the appropriate peak time difference adjustments made based on the calibration data.

According to exemplary data, at a 256 Hz sampling rate with no inherent time difference between true beats, the time difference probability for true beats falls off sharply from a 0 sample difference to a 2 sample difference, with very low probabilities for time differences greater than 2 samples.

The peak coherence measure is reduced to the extent that the signals from two channels are highly correlated, but not so highly correlated (e.g. correlation coefficient >0.6) that the signals are averaged together as described with reference to block 103 of FIG. 2. In particular, in the case of two correlated channels, the measure is scaled by 1−min(1,3*max(0,max(cormat(:))−0.3)), where cormat is a matrix that of correlation coefficients between the channels. In the case of more than two channels, a linear combination of scaling factors is preferably applied to the coherence measure.

FIG. 7 is a flow chart of the process for computing a sequence's raw score. Blocks 702, 704 and 706 apply various corrections to the temporal regularity measure (TR). Block 702 penalizes high TR score sequences by further decreasing their TR scores. This correction reduces the chance that relatively more irregular sequences will be selected unless they tend to have greater peak coherence and/or peak prominence measures. Block 704 penalizes low RR interval (high heart rate) sequences by decreasing their TR scores. This correction reflects the relatively greater regularity of true high heart rate sequences. Block 706 rewards high RR interval (low heart rate) sequences by increasing their TR scores. This correction reflects the greater tendency of true low heart rate sequences to be temporally irregular.

Block 708 computes the peak pair prominence quality measure PPS for a sequence by summing its pertinent entries in the global peak pair prominence matrix PP. Block 709 decreases the PPS of low RR interval sequences, which would otherwise tend to have high PPS due to the relatively greater number of peak pairs in these sequences. (Alternatively, PPS could be normalized by the number of peak pairs in a sequence.) Block 710 sets the raw score of a sequence as a weighted sum of: 1) the sum of the peak coherence probabilities for all peaks in the sequence that are parent peaks; 2) PPS; 3) the sequence's temporal regularity quality measure; and 4) the number of skipped beats in the sequence. The weights are preferably determined by performing a least squares fit to a sequence evaluation measure (e.g. accuracy or F1) that is known for training data.

In an alternative embodiment, instead of using a probability distribution of any sort in the first sum element (peak time coherence), a simple increasing function inversely related to peak time differences could be applied. An example of such a function is max((3−x)/3,0), where x is peak time difference in samples at a sampling rate of 256 Hz.

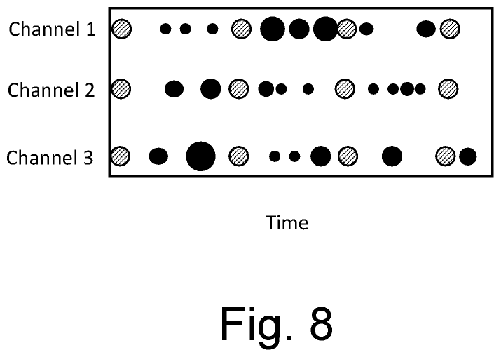

FIG. 8 is an image of peaks, mapped by size/quality into circles, that may be used to train a neural network to perform peak detection according to the teachings of the present invention. For purposes of illustration, the true peaks as shown with diagonal stripes while noise peaks are filled solid black.

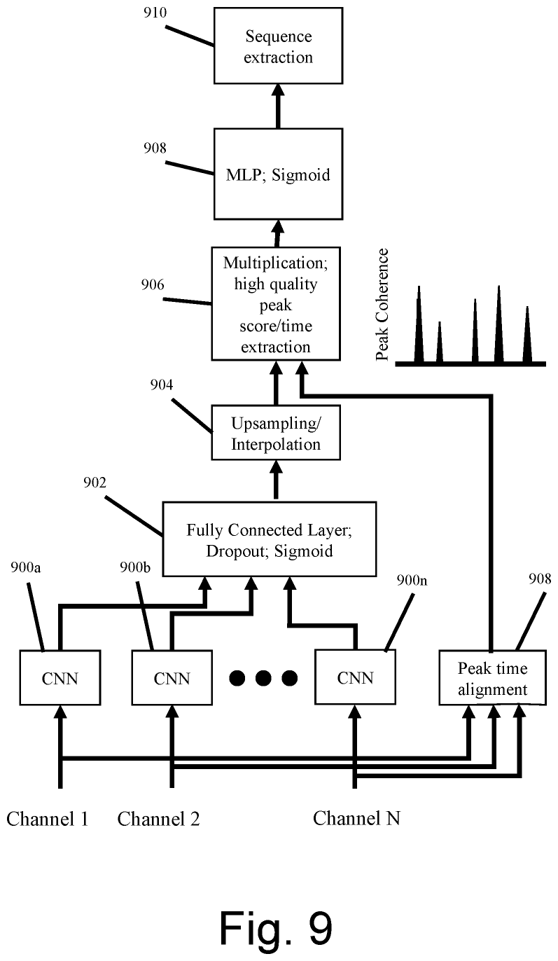

FIG. 9 is a block diagram of a convolutional neural network implementation of the present invention. Signals corresponding to N channels are separately processed by a corresponding one of a plurality of convolutional neural networks (CNN) 900a, 900b, . . . , 900n. A possible architecture for each of the CNNs 900a, 900b, . . . , 900n is described in Cai, W.; Hu, D. “QRS Complex Detection Using Novel Deep Learning Neural Networks” IEEE Access 2020, 8, 97082-97089. The output of the CNN's 900 is a downsampled indicator signal with a value between 0 and 1 according to the probability that a peak exists.

These indicator signals are provided to block 902, which comprises a fully connected layer, followed by a dropout layer and a sigmoid layer, which outputs a combined indicator signal that is a downsampled indicator signal with a value between 0 and 1 according to the probability that a peak exists. In block 904, the indicator signal is upsampled/interpolated so that it has the same number of samples as the signals input to the CNNs 900 and a peak time alignment module 908.

The peak time alignment module 908 implements the methods described with reference to FIG. 6. The output of module 908 is a peak coherence score that has the value from FIG. 6, block 604 at the global peak locations and a fraction of this value that linearly decreases with distance (number of samples) from the global peak locations. The triangular output is depicted adjacent to block 906 in FIG. 9. The width of the base of the triangle is an increasing function of the dispersion of the local peak tines corresponding to a global peak.

Block 906 multiplies the outputs from blocks 904 and 908 and extracts the resulting scores. The highest quality peak scores, and the resulting peak times, are provided to a multilayer perceptron 908, which applies a final sigmoid activation layer to output a function indicative of peak quality. Block 910 extracts a winning sequence from the peak quality function by performing a sequence search and optimizing the sum of peak quality from MLP 908.

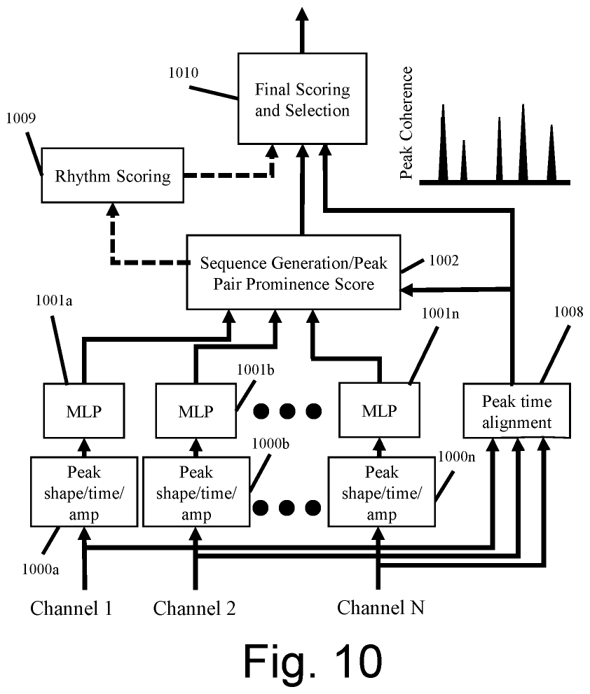

FIG. 10 is a block diagram of a feature extraction based partial neural network implementation of the present invention. Signals corresponding to N channels are separately processed by a corresponding one of a plurality of feature extraction modules 1000a, 1000b, . . . , 1000n, which perform peak detection and obtain peak shape, amplitude and time information. The peak shape information comprises the amplitudes and durations of the (possible) Q, R and S portions of potential heartbeat peaks.

The peak shape/time/amplitude information from each of the feature extraction modules 1000a, 1000b, . . . , 1000n is provided to a corresponding one of a plurality of multi-layer perceptrons (MLPs) 1001a, 1001b, . . . , 1001n, which determine peak quality measures based on the peak amplitude/shape information. In an alternate embodiment, peak shape/amplitudes are clustered, and the quality of the clusters determines the peak shape/amplitude quality of the peaks.

Each of the MLPs 1001a . . . n provides peak times and associated quality measures to sequence generation block 1002, which also receives global peak time information from block 1008, which preferably implements the peak time alignment steps described with reference to FIGS. 3a and 3b. In an alternate embodiment, block 1008 is a convolutional neural network that cross correlates data across channels over time periods on the order of the peak width of the heartbeats to be detected. Such a network can be trained with multi-channel signals with true beats separated by known time periods, with the known output set to 1 at the mean of the true peak times and 0 elsewhere. Alternatively, the output could be set equal to a triangular or rectangular function with centered at the mean of the true peak times but with 0 values outside of a window consistent with the probability density function of true peak time differences as discussed above with reference to block 604 of FIG. 6. For human QRS complexes, this window is preferably 4 ms-12 ms, and in any event less than one half of the typical QRS duration.

The sequence generation block 1002 maps local peaks to global peaks according to the peak time alignment information, generates sequences from high quality peak pairs, and sums the peak pair prominence scores from MLPS 1001a . . . n for each sequence.

Sequence time information is provided by block 1002 to block 1009, which provides a rhythm score. The rhythm score is a temporal regularity score (as previously described with regard to block 316 of FIG. 3) for sinus rhythm, and a probability score for regular abnormal rhythms (e.g. bigeminal).

Block 1010, which generates final sequence scores and sequence selection, is coupled to blocks 1008, 1002 and 1009. The dashed lines connecting block 1009 to blocks 1009 and 1010 indicate that the rhythm score is applied only to the extent sequence rhythms are probabilistically distinguishable from noise. If all of the inputs to block 1010 are scaled such that they indicate true probability measures, block 1010 simply implements standard probability summing rules. Otherwise, block 1010 can implement Sugeno or Choquet integrals to sum quality measures.

The MLPs 1001a . . . n are preferably configured to output a quality score based solely on peak prominence and shape characteristics (of both noise and cardiac signals). Thus, the MLPs 1001a . . . n are preferably trained on a variety of noise data and with cardiac signals characterized by a wide variety of rhythms, including atrial fibrillation. Direct connections in the MLPs 1001a . . . n preferably extend only between peaks that are physiologically possible consecutive heartbeat pairs.

The methods and architectures described above with reference to FIGS. 2-10 involve solving a combinatorial optimization problem. A graph neural network, which doesn't necessarily separate sequence generation from scoring, could also be used to detect heartbeats according to the teachings of the present invention.

What is claimed is:

1. A method for detecting heartbeats comprising the steps of:
a. receiving first and second cardiac signals from a plurality of sensors;
b. recording the first and second signals and identifying time segments within a recording;
c. detecting a first set of peaks within a first segment in the first cardiac signal to comprise a first set of peaks;
d. detecting a second set of peaks within a second segment in the second cardiac signal to comprise a second set of peaks, the first and second segments being at least partially contemporaneous;
e. generating a third set of peaks comprising:
i. at least one merged peak identified by grouping peaks from the first and second sets of peaks according to time of occurrence; and/or
ii. at least one peak selected from a set that includes the union of the first and second sets of peaks;
f. searching within the third set of peaks for permissible peak sequences such that each of at least two permissible peak sequences includes a peak that is not in the other sequence;
g. determining a quality score for each of the permissible peak sequences;
h. selecting one of the permissible peak sequences according to its score, thereby detecting a plurality of putative heart beats.

2. The method of claim 1 wherein the quality score is based on a peak timing coherence quality measure, wherein the peak timing coherence quality measure discriminates between peak time differences of true beats and noise peaks.

3. The method of claim 1 wherein the quality score is based on peak prominence.

4. The method of claim 1 wherein the quality score is based on heart rhythm.

5. The method of claim 4 wherein the heart rhythm is sinus rhythm.

6. The method of claim 1 wherein the quality score is based on a peak shape feature.

7. The method of claim 1 wherein the step of searching within the third set of peaks comprises the steps of:
a. selecting a set of high-quality peaks from the third set of peaks;
b. generating parent subsequences from the set of high-quality peaks; and
c. for at least some of the parent subsequences, generating an offspring sequence by searching for peaks that fill gaps within the corresponding parent subsequence.

8. The method of claim 1 wherein the step of searching within the third set of peaks comprises the step of finding peaks that form a temporally acceptable sequence with a peak in a prior segment.

9. The method of claim 8 wherein the prior segment does not overlap in time with either the first or second segments.

10. The method of claim 1 wherein the quality score is based on a sequence's raw score and a raw score from a temporally matching sequence from a prior and/or subsequent segment.

11. The method of claim 1 wherein the first signal is an electrocardiographic signal.

12. The method of claim 11 wherein the second signal is an electrocardiographic signal.

13. The method of claim 1 wherein the quality score for a candidate sequence is determined by summing output values indicative of peak quality from a neural network, wherein each of the output values corresponds to a peak within the candidate sequence.

14. A method for detecting heartbeats comprising the steps of:
a. receiving first and second cardiac signals from a plurality of sensors;
b. recording the first and second signals and identifying time segments within a recording;
c. detecting a first set of peaks within a first segment in the first cardiac signal to comprise a first set of peaks;
d. detecting a second set of peaks within a second segment in the second cardiac signal to comprise a second set of peaks, the first and second segments being at least partially contemporaneous;
e. generating a third set of peaks comprising:
i. at least one merged peak identified by grouping peaks from the first and second sets of peaks according to time of occurrence; and/or
ii. at least one peak selected from a set that includes the union of the first and second sets of peaks;
wherein at least two peaks in the third set of peaks are separated by a physiologically impermissibly short time period;
f. searching within the third set of peaks for permissible peak sequences;
g. determining a quality score for each of the permissible peak sequences;
h. selecting one of the permissible peak sequences according to its score, thereby detecting a plurality of putative heart beats.

15. A method for detecting heartbeats comprising the steps of:
a. receiving first and second cardiac signals from a plurality of sensors;
b. recording the first and second signals and identifying time segments within a recording;
c. detecting a first set of peaks within a first segment in the first cardiac signal to comprise a first set of peaks;
d. detecting a second set of peaks within a second segment in the second cardiac signal to comprise a second set of peaks, the first and second segments being at least partially contemporaneous;
e. generating a third set of peaks comprising:
i. at least one merged peak identified by grouping peaks from the first and second sets of peaks according to time of occurrence; and
ii. at least one non-merged peak selected from a set that includes the union of the first and second sets of peaks;
f. searching within the third set of peaks for permissible peak sequences;
g. determining a quality score for each of the permissible peak sequences;
h. selecting one of the permissible peak sequences according to its score, thereby detecting a plurality of putative heart beats.

16. A method for detecting heartbeats comprising the steps of:
a. receiving N cardiac signals from a plurality of sensors, wherein N is greater than 1;
b. determining a non-binary peak timing coherence quality measure from the N cardiac signals, wherein the peak timing coherence quality measure discriminates between peak time differences of true beats and noise peaks; and
c. selecting a particular sequence of peaks according to an optimal sequence quality score based on the non-binary peak time coherence quality measure, thereby detecting a plurality of putative heart beats.

17. The method of claim 16 wherein the non-binary peak time coherence quality measure is based on an a priori probability distribution.

18. The method of claim 17 wherein the a priori probability distribution is estimated by adding calibrated amounts of noise to a clean signal and comparing the statistical properties of noise peaks with heartbeat peaks.

19. The method of claim 17 wherein the a priori probability distribution is a function of noise level.

20. The method of claim 17 wherein the a priori probability distribution is a function of N.

21. The method of claim 17 wherein the a priori probability distribution is a function of heart rate.

22. The method of claim 17 wherein the non-binary peak time coherence quality measure is determined by detecting a set of peaks in each of at least two of the cardiac signals, determining peak times associated with each of the peaks, deriving peak time differences between peaks in the different sets of peaks, and obtaining values of the a priori probability distribution as a function of the peak time differences.

23. The method of claim 16 wherein the non-binary peak time coherence quality measure pertains solely to peak time differences.

24. The method of claim 16 wherein the non-binary peak time coherence quality measure is determined by a neural network.

25. The method of claim 16 wherein the sequence quality score is further based on peak prominence.

26. The method of claim 16 wherein the sequence quality score is further based on heart rhythm.

27. The method of claim 16 wherein the optimal sequence quality score is determined by comparing sequence quality scores explicitly determined for corresponding sequences.

28. The method of claim 16 wherein at least 2 of the N cardiac signals are electrocardiograms.

29. A system for detecting inter-peak intervals in a plurality of cardiac signals, comprising:
a. a sensing apparatus comprising a plurality of sensors adapted to receive at least two cardiac signals,
b. a processing apparatus adapted to: (i) receive first and second cardiac signals from the plurality of sensors; (ii) record the first and second signals and identifying time segments within a recording; (iii) detect a first set of peaks within a first segment in the first cardiac signal to comprise a first set of peaks; (iv) for each peak within a first subset of the first set of peaks, determine a peak quality measure; (v) detect a second set of peaks within a second segment in the second cardiac signal to comprise a second set of peaks, the first and second segments being at least partially contemporaneous; (vi) for each peak within a second subset of the second set of peaks, determine a peak quality measure; (vii) perform combinatorial optimization on a third set of peaks that includes the first and second subsets, wherein the combinatorial optimization is based on the peak quality measures corresponding to the first and second subsets respectively; (viii) generate inter-peak interval information based on the combinatorial optimization;
c. a display apparatus for receiving from the processing apparatus information based on the inter-peak interval information.

30. The system of claim 29 wherein the inter-peak interval information pertains to QRS complexes.

31. The system of claim 29 wherein the processor is configured to generate sequences of peaks from the first and second sets of peaks, and to select a particular sequence upon which the interpeak interval information is based.

32. The system of claim 29 wherein the combinatorial optimization is further based on timing coherence between peaks within the first and second sets respectively.

33. The system of claim 29 wherein the processor is configured to merge corresponding peaks from the first and second sets of peaks, thereby generating merged peaks and possibly non-merged peaks.

34. The system of claim 29 wherein the peak quality measure is based on peak prominence.
